(19) United States
(12) Patent Application Publication
(10) Pub. No.: US 2004/0162562 A1
(43) Pub. Date: Aug. 19, 2004

(54) Instrumentation system for treating end plates of adjacent vertebrae

(75) Inventor: Erik O. Martz, Howell, NJ (US)

Correspondence Address: YURI KATESHOV, ESQ., DILWORTH & BARRESE, LLP, 333 Earle Ovington Blvd., Uniondale, NY, 11553, US

(73) Assignee: OSTEOTECH, INC., Eatontown, NJ

(21) Appl. No.: 10/366,181

(22) Filed: Feb. 13, 2003

(51) Int. Cl. A61B 17/16

(52) U.S. Cl. 606/79

(57) ABSTRACT

An instrumentation system, configured to place adjacent vertebrae at a fixed distance and desired spatial position relative to each other, includes a guide frame guiding an endplate preparation instrument into a disc space between the opposing endplates of the adjacent vertebrae so that the treated surfaces of the endplates define the disc space of the desired shape.

### BACKGROUND

1. Field of the Invention

The invention relates to a guide and end plate preparation instrumentation system configured to prepare opposing end plates of adjacent vertebrae so as to form a pocket, shaped and dimensioned to correspond to the shape and dimensions of the selected spinal implant.

2. Background of Related Art

The spine is a flexible column formed of a series of bones called vertebrae. The vertebrae are hollow and piled one upon another, forming a strong hollow column for support of the cranium and trunk. The hollow core of the spine houses and protects the nerves of the spinal cord. The different vertebrae are connected together by means of articular processes and intervertebral, fibro-cartilages.

The intervertebral fibro-cartilages are also known as intervertebral discs and are made of a fibrous ring filled with pulpy central material. The discs function as spinal shock absorbers and also cooperate with synovial joints to facilitate movement and maintain the flexibility of the spine. When one or more discs degenerate through accident, spondylolisthesis or other pathologies, nerves passing near the affected area may be compressed and are consequently irritated. The result may be chronic and/or debilitating back pain. Various methods and apparatus, both surgical and non-surgical, have been designed to relieve such back pain.

One method designed to relieve such back pain is interbody spinal fusion. Typically, interbody spinal fusion involves distracting adjoining vertebrae of the spine so that the nerve root canal sizes are increased and nerve irritation is eliminated or reduced. In order to maintain the adjoining vertebrae of the spine in a distracted state, at least one intervertebral implant is inserted into a receiving bed formed between the vertebrae. The implant is positioned to engage the adjoining vertebrae to maintain the vertebrae at a fixed degree of distraction. Preferably, the implant should stabilize the intervertebral space and become fused to adjacent vertebrae in order to prevent the implant and adjacent vertebrae from moving.

A variety of different types of intervertebral implants have been developed to perform this function including spinal fusion cages, threaded bone dowels, stepped bone dowels and wedges.

In order to install these implants the disc space and often the faces of the end plates of the adjacent vertebrae need to be prepared to receive the implant by removal of disc material and/or forming a bed in the faces of the end plates. These operations are typically carried out by an operating surgeon making decisions based on his/her experience. Accordingly, in a large number of instances, the opposing end plates defining a pocket therebetween are not uniformly shaped, and thus may not be placed in the desired spatial relationship, which should correspond to the known shape of the selected spinal implant.

Accordingly, a need exists for a system of instrumentation and a surgical method for advancing, positioning and operating end plate preparation instruments so as to create an implantation disc space provided with a shape and dimensions corresponding to the selected shape and dimensions of the selected implant

### SUMMARY OF THE INVENTION

Consonant with the above-formulated objective, an instrumentation system for preparing end plates of adjacent vertebrae to be fused includes a frame, insertable into a distracted disc space defined between adjacent end plates, and end plate preparations instruments displaceable into the disc space along a uniform path defined by the frame. The uniform path allows multiple end plate preparation instruments including, but not limited to, chisels and rasps, to be so oriented within the disc space that the opposing end plates of the vertebrae to be fused are aligned in the desired spatial position. The inventive system and method eliminate free hand motions to form the disc space characterized by the precise size, shape and configuration, as well as to make the extent of the vertebral endplate resection predictable, precise and reproducible, if necessary.

Thus, the primary advantage of the present inventive system is its ability to uniformly prepare the opposing end plates of the adjacent vertebrae to be fused in the desired anatomical conformation leading to the formation of the disc space corresponding to the selected shape of the spinal implant. As a result, the inventive system allows for the maximum stability of the selected spinal implant, as well as the construct, by providing for the optimal interface surface area and congruency between the implant and each of the opposing end plates of the vertebrae to be fused.

According to one aspect of the present invention, the frame is configured with a combination of stops preventing uncontrollable penetration of the end plate preparation instruments into the disc space, as well as determining a desired engaging position of the frame and the adjacent vertebrae. The geometry of the frame allows the stops to be placed at strategic locations along the paths of the frame, displaceable relative to the vertebrae, and to controllably stop displacement of the frame and the instruments into the disc space. In addition to the stops, this aspect of the invention is concerned with a guide assembly provided between the frame and the end plate instruments and defining a desired path of the end preparation instruments into and out of the disc space.

Accordingly, one of the advantages of the inventive instrumentation system is that it can be selected and calibrated for the maximum safe penetration depth of the frame into the disc space and for locking the instruments at the desired depth so as to prevent unwanted over penetration of these instruments.

A further aspect of the invention is directed to a modular structure of the frame configured to be piecemeally disassembled in its inserted position within the treated disc space to facilitate subsequent insertion of a spinal implant. While different combinations of frame components can be removed from the disc space, the remaining component(s) of the frame would still maintain the disc space as prepared by the end plate instruments.

Still another aspect of the invention includes a variety of end preparation instruments each provided with a structure that cooperates with the inventive frame to shape the end plates so that they would be in desired alignment. In particular, the structure of the inventive end plate preparation instruments is configured to have guide surfaces mating with the guides and stop formations of the frame so that the end plates are prepared to define therebetween an implant receiving pocket formed with the desired length and depth.

A further aspect of the invention relates to a surgical kit including the inventive frame, end plate preparations instruments, specifically configured to cooperate with the frame, and a plurality of implants of different sizes and shapes. In addition, the kit may include a combination of actuating elements, such as various tools and implant holders as well as slap hammers facilitating the removal of the inserted system components from the disc space.

It is an object of the invention to provide an instrumentation system for use in spinal surgery configured to advance, position and operate a plurality of end plate preparation instruments so as to create the implantation space of the selected shape and dimensions corresponding to those of the known implants.

Another object of the invention is to provide a guide including a frame, which is configured, to be inserted into the distracted disc space so as to provide for the controllable advancement of the end plate preparation instruments into the disc space.

Still another object of the invention is to provide the frame having a modular structure configured to allow variously shaped and dimensioned spinal implants to be inserted between the prepared end plates.

A further object of the invention is to provide a set of end plate preparation instruments configured to cooperate with the frame and shaped to controllably deepen and elongate the distracted disc space.

Another object of the invention is to provide a kit including a combination of instrument guides and end plate instruments configured in accordance with the invention.

### BRIEF DESCRIPTION OF THE DRAWINGS

The above and other objects, features and advantageous will become more readilly apparent from the following specfic description dsiclosed in conjunction with a set of drawings, in which:

FIG. 1 is a perspective view of an instrumentation system configured in accordance with the invention and shown in a deployed position of a chisel;

FIG. 2 is an isometric view of an end plate preparation instrument;

FIG. 3 is a perspective view of an end plate praparation instrument guide of the instrumentation system shown in FIG. 1;

FIG. 4 is a top plan view of the instrument guide of FIG. 2;

FIG. 5 is an end view of an end plate preparation instrument of FIG. 2;

FIG. 6 is an exploded perspective view of the instrument guide of FIG. 2 having a modular structure;

FIG. 7 is an exploded perspective view of the instrumentation system configured in accordance with another embodiment of the invention;

FIG. 8 is a side view of a slap hammer for use with the instrument guide of FIGS. 3 and 8. indicia

FIG. 9 is a perspective view of another inventive embodiment of the chisel guide; and

FIG. 10 is perspective view of an end plate preparation instrument configured for use with the chisel guide of FIG. 9.

### DETAILED DESCRIPTION OF PREFERRED EMBODIMENT

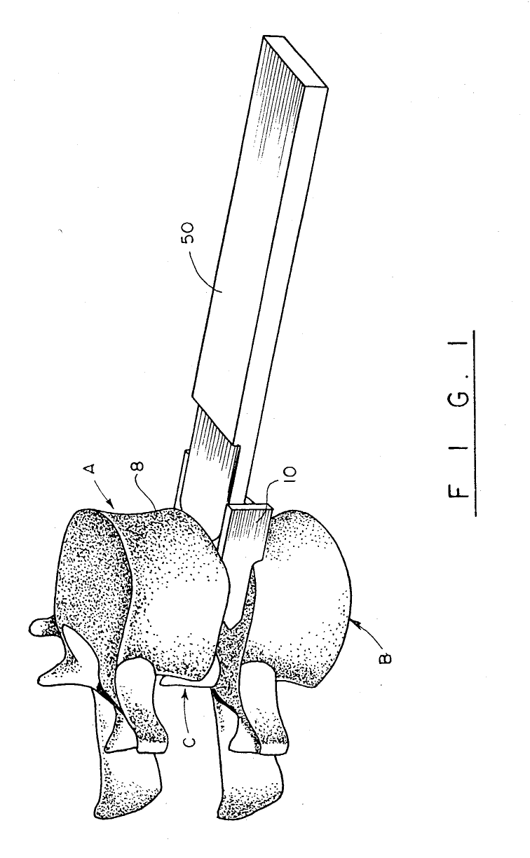

The present disclosure relates to various embodiments of an instrumentation system for use in intervertebral implant procedures directed to preparation of a pocket defined between end plates of adjacent vertbrae A and B and configured to receive a spinal implant. Referring initially to FIGS. 1-5, and particualrly to FIG. 1, the instrumentation system includes an instrument guide 10, configured to be inserted into a disc space C between opposing end plates of the adjacent vertebrae A and B, and an end plate preparation instrument 50 displaceable along the the guide 10 into the disc space C.

The inventive instrumentation system provides for controllable penetration of the instrument guide 10 and the end plate preparation instrument 50 into the disc space C along the desired uniform path of the end plate instruments. Accordingly, the end plate preparation instruments remove the same amount of material from and apply the uniform shape to the opposing upper and lower end plates of the adjacent vertebrae A and B. Hence, the disc space, thus prepared, conforms to the selected shape of the known spinal implant, which is critical to the safe and successfult outcome of the spinal surgery. The following discussion will be directed to the end plate preparation instrument 12 which, for purely illustrative purposes, includes a chisel. However, other end plate preparation instruments can be used in combination with the instrument guide 10, provided these instruments are configured so as to cooperate with the guide's strcuture in a controllable manner limiting the instruments' penetration into the disc space along the desired path defined by the guide.

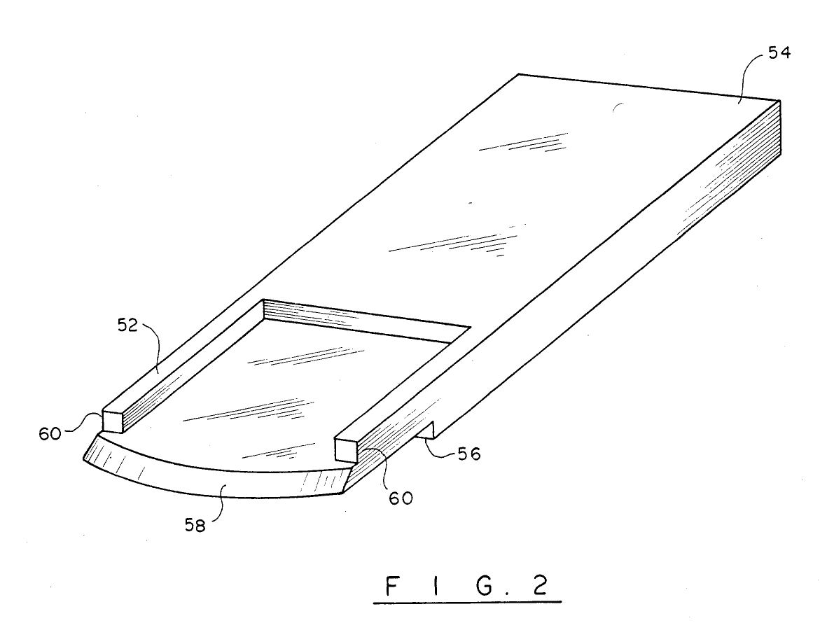

Referring now to FIG. 2, the chisel 50 includes a front portion 52 configured to be inserted into the disc space C and a handle portion 54 extending rearwards from and having a thickness larger than a thickness of the front portion 52. Accordingly, the front 52 and handle 54 portions of the chisel 50 define therebetween a rear stop 56 configured to abut an outer surface 8 (FIG. 1) of either upper A or lower B vertebra to prevent uncontrollable penetration of a cutting edge 58 of the chisel 50 into the disc space. Spaced frontward from the rear stop 56 is front stops 60 configured to abut formations on the instrument or chisel guide 10 to ensure that the cutting edge 58 would not advance into the disc space father than a depth required by the dimensions of the selected spinal implant. Other configurations of the front stops 60 are envisioned and may include, for example, instead of two spaced stops, a continuous front stop. A height of the stop(s) 60 cooperating with a depth of chisel guide 10, as will be explained further, determines the appropriate amount of depth of cut into the vertebrae.

Turning to FIGS. 3-5, the chisel guide 10 generally includes a frame provided with a center shelf 12 and a pair of opposing sidewalls 14 and 16. A forward wall 18 extends between the distal ends of sidewalls 14 and 16. Preferably forward wall 18 has an arcuate shape facilitating insertion of chisel guide 10 between adjacent vertebrae. The combination of center shelf 12 and sidewalls 14 and 16 define a cumulative depth D of two pockets formed in the upper and lower end plates, respectively, and substantially corresponfing to the height of the sidewalls, and a width W, corresponfing to a distance between the inner sides of the sidewalls, which is selected to receive the chisel 50. Chisel guide 10, as illustrated in FIGS. 3-5, is monolithically formed of material such as stainless steel.

Sidewalls 14 and 16 are provided with upper and lower stops 20 and 22, respectively, (FIG. 5) at proximal ends of the sidewalls. Stops 20, 22 can have spikes 23, formed either integrally therewith or mounted on the stops through holes (not shown) after the guide 10 is fully inserted onto the disc space. The spikes are configured to penetrate the vertebrae and secure the chisel guide to the vertebrae. Stops 20 and 22 function to engage the outer surface 8 (FIG. 1) of the vertebrae and limit the depth of insertion of chisel guide 10 between the adjacent vertebrae. Sidewall 14 has an upper vertebral supporting surface 24 and a lower vertebra-supporting surface 26, whereas sidewall 16 has an upper vertebral supporting surface 28 and a lower vertebral supporting surface 30. Forward wall 18 has a chamfered nose extending between edges 32 and 34. Upper supporting surface 24 and lower supporting surface 26 of sidewall 14 as well as upper supporting surface 28 and lower supporting surface 30 of sidewall 16 are configured to engage and support adjacent vertebrae in a distracted condition during the entire chiseling operation. The edges 32 and 34 of forward wall 18 only support the adjacent vertebrae during initial insertion and cutting of the chisel 50. Thereafter, the disclosed chisel will extend above and beyond upper edge 32 or lower edge 34 of forward wall 18 depending upon which vertebrae is being cut.

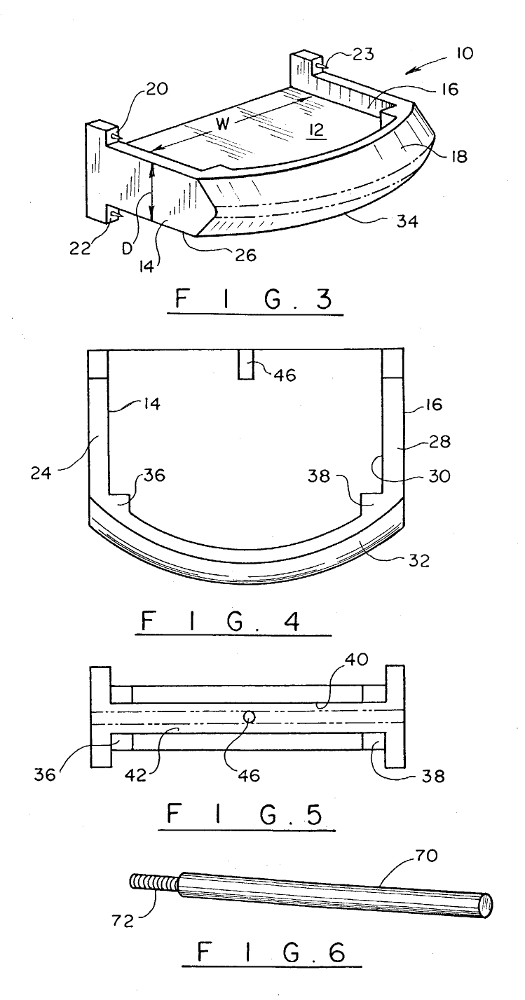

Preferably, chisel guide 10 includes upper and lower stops 36, at the intersection of sidewall 14 and forward wall 18 and upper and lower stops 38 formed at the intersection of sidewall 16 and forward wall 18. Stops 36 and 38 are provided to selectively engage the front stops 60 on the chisel 50, depending upon which vertebrae is being treated, and to limit the depth of penetration of the chisel within the vertebral disc space. Center shelf 12 includes an upper guiding surface 40 and a lower guiding surface 42 to provide a platform against which the chisel 50 can slide. As shown in FIGS. 4 and 5, chisel guide 10 further includes a bore 46 formed in a proximal edge of shelf 12 for receipt of an insertion tool. The bore 46 may or may not be threaded depending on the structure of the insertion tool.

In order to provide room for chisel guide 10, adjacent vertebrae A and B, defining therebetween the collapsed disc space C, need to be forced apart or distracted by a distractor typically having a distraction head and a shaft extending therefrom. The distractor is inserted between vertebrae A and B and force or wedge apart vertebrae A and B. This can be done in a single step or with multiple distractors serially distracting from smaller to larger heights until the appropnate tension between the adjacent vertebrae is achieved. Insertion of the distractor continues until the distraction head is fully seated between the adjacent vertebrae at the desired height. Incidentally, the chisel guide 10 can be used as a distractor by gradually introducing the front, generally frustoconical forward wall 18 between the adjacent vertebrae, which, thus, are displaced away from one another to reach the depth D of the guide defined between opposite supporting surfaces 24, 26 and 28, 30 (FIG. 3).

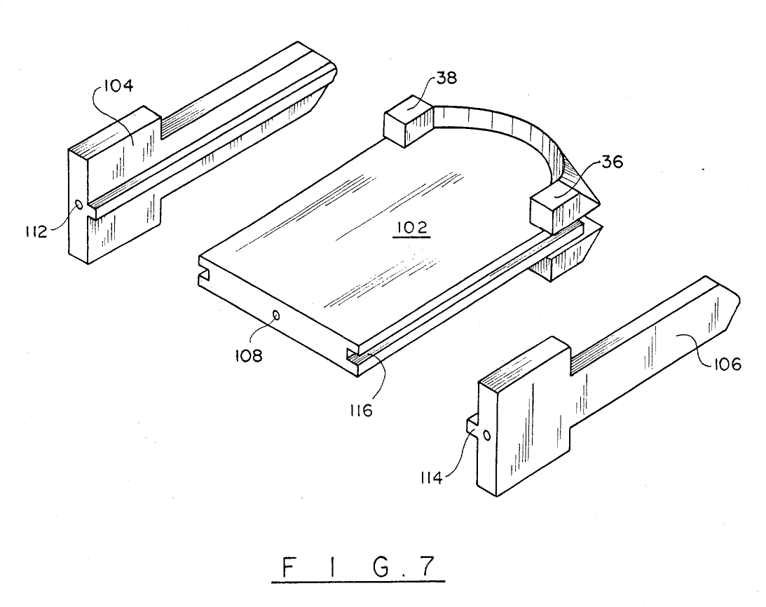

Turning to FIG. 6, to insert chisel guide 10 between the now distracted vertebrae, an insertion tool 70 having a threaded pin 72 is attached to chisel guide 10 by threading in the pin 72 into threaded bore 46 (FIG. 5). In response to a thrust generated by the operator, insertion tool 70 advances chisel guide 10 into the distracted disc space C at the depth of penetration defined by rear stops 20 and 22 of the chisel guide 10 engaging the outer surface 8 (FIG. 1) of the adjacent vertebrae. The stops 20, 22 are so located that the chisel guide 10 is fully inserted between the adjacent disc space when the stops abut this outer surface of the vertebrae, after which insertion tool 70 can be unthreaded.

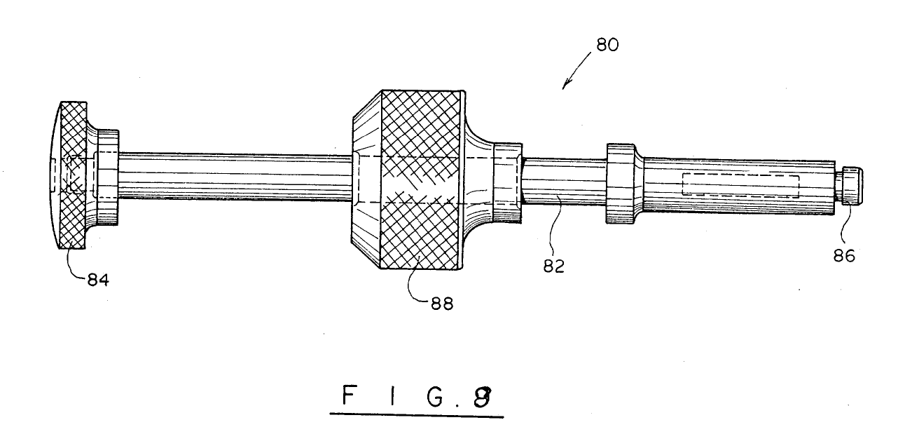

At this point, as illustrated in FIG. 1, the chisel 50 is introduced into the upper or lower compartment of the chisel guide 10, each of which is defined between the forward wall 18, side walls 14 and 18 and either upper 40 or lower 42 guiding surface of the shelf 12. The chisel 50 and the compartments are configured so that the front portion 52 of the chisel is slidingly guided by respective surfaces of the sidewalls 14, 16 and the shelf 12 of the chisel guide 10 without deviations from the desired path. Accordingly, since the paths along the upper and lower compartments are identical and the shelf 12 extends substantially midway between the opposing end plates, the chisel 50 removes substantially the same amount of material from the end plates each of which is, thus, prepared with a substantially identical pocket. Accordingly, the disc space has the desired shape and dimensions corresponding the shape and dimensions of the selcted implant. The inner surfaces of the sidewalls 14, 16 and opposing outer surfaces of the chisel 50 may have variously shaped detents and projections 78 (FIG. 7) slidingly engaging one another to even further ensure the uniformity of the path along which the chisel guide 50 slides relative the guide 10. Furthermore, each of the sidewalls 14 and 16 may be formed with a dovetail shape necessitating the slanted sides of the chisel 50. The chisel guide could also be hollow; i.e., its shelf could have a window to allow for passage of debris during endplate preparation. This debris could then be removed on the underside of the guide.

Advancement of the chisel 50 into the disc space continues until front the stop(s) 60 engage the upper or lower stops 36, 38 and/or the rear stop 56 engages the outer surfaces 8 of the vertebra to prevent any further insertion of chisel 50 into the disc space C. Once one of the lower or upper vertebra has been cut and its end plate prepared, chisel 50 can be removed and reinserted to cut the other vertebra in similar fashion.

The prepared disc space includes pockets formed by removing the substnatially uniform amount of material in both the upper vertebrae and lower vertebrae. Note that the geometry of the chisel 50 allows the cutting edge 58 to extend beyong the forward side 18 of the shisel guide 10 while the upper 24, 28 or lower 26, 30 supporting surfaces of the sidewalls 14, 16, respectively, support the adjacent end plates of the vertbrae A and B. Accordingly, since the length of the cut in the vertebrae extends beyond the distal end of chisel guide 10, the upper supporting surface 32 and lower supporting surface 34 of forward wall 18 no longer support the adjacent vertebrae as the material has been cut away beyond this ledge of chisel guide 10.

Once the chiseling operation has been completed, insertion tool 70, as seen in FIG. 6, can be used to remove the chisel guide 10 from the disc space. It may be useful to provide a slap hammer 80 as shown in FIG. 8 to facilitate withdrawal of chisel guide 10 and attached insertion tool 60. Slap hammer 80 generally includes an elongated shaft 82 having an end cap 84 threaded on a proximal end thereof. Attachment structure 86 is provided at a distal end of shaft 82 and is configured to engage proximal end of the insertion tool 70. A movable weight 88 is provided between the proximal distal ends of slap hammer 80 so as to facilitate withdrawal of insertion tool 60 by forcing weight 88 approximately against end cap 84. Upon removal of at least part of the guide 10, the prepared end plates have a contour and dimesnions conforming to thise of the selected spinal implant.

Referring then to FIG. 7, there is illustrated a modular structure of the chisel guide 10 having the first component consisting of center shelf 102, the second component including sidewall 104 and the third component consisiting of another sidewall 106. The sidewalls 104 and 106 are removably attached to center shelf 102. Thus, guide tool 10 is provided with three separable components each of which, once the appropriate cuts have been made in the adjacent vertebrae, may be selectively removed from the prepared disc space C position. At least one of the side walls 104, 106 may remain within the disc space to maintain the appropriate spacing and distraction of the adjacent vertebrae A and B.

Center shelf 102 includes a bore 108 for receipt of an insertion tool and similarly sidewall 106 includes a bore 112 for receipt of a similar insertion tool. Each of the sidewalls 1104, 106 includes a projection 114 which is configured to engage slot 116 fromed in the sidewalls of the center shelf 102. The location of the prjections and slots can be reversed so as to have the shelf 12 formed with projections 114 engaging the slots 116, which would be formed on the sidewalls 104, 106. Preferably, the projections and slots 116 are of the dovetail variety. Insertion tool 70 (FIG. 6) can be selectively inserted in bores 108, 112 of center shelf 102 and sidewalls 104 and 106, respectively, to remove any of these from between the adjacent vertebrae. The side walls would be placed first, by inserting them in their “small” state and then rotating them 90 degrees until they are in the orientation shown in FIG. 7, then the center piece 102 would be inserted. The side pieces can have means to attach other inserter/handles that allow for translational insertion plus rotation into final position. Alternatively, the guide may be formed as a two part chisel guide consisting of a center shelf 102 and one of the sidewalls 104, 106 fromed integrally with the shelf. In a still another alternative configuration of the ebntire system, the stops 36, 38, located on the shelf 102, can be formed on the inner surfaces of the sidewalls 104, 106.

In use, once the end plate is shaped and dimensioned as desired, the shelf is removed freeing a sufficient space to insert the implant between the sidewalls, which, in turn, can be removed after the implant is properly positioned. Either of the sidewalls may removed simultanoeusly with the shelf 102 to provide additional clearance for insertion of the selected implant.

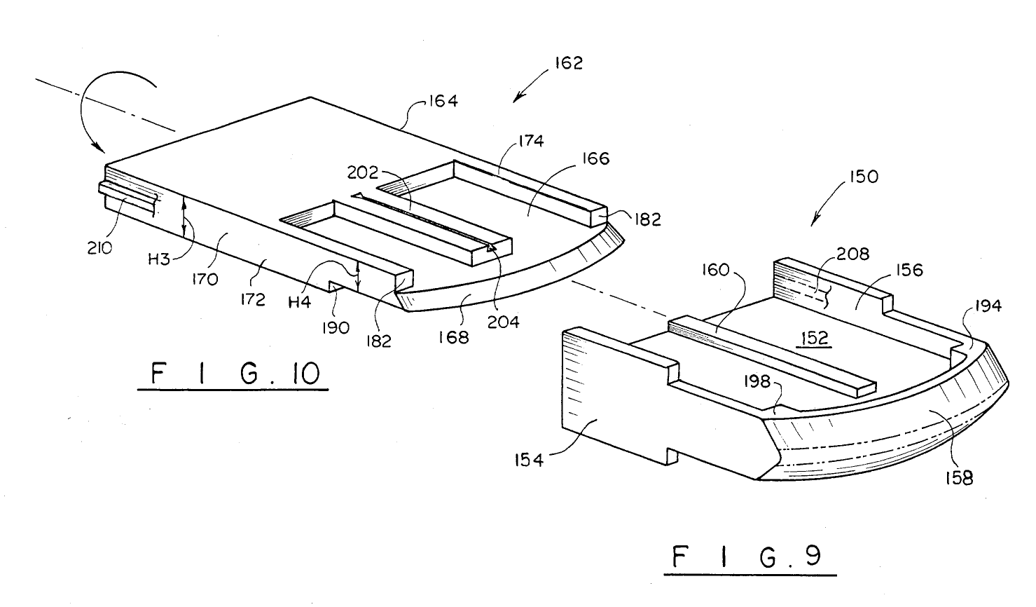

Referring now to FIGS. 9-10, there is illustrated further embodiment of a chisel guide 150 and an associated chisel 162 for use in forming and preparing end plates of adjacent vertebrae. The disclosed chisel 162 and chisel guide 150 are configured to matingly engage with each other so as to provide support and guidance for the chisel 162 during the cutting operation. Referring specificaly to FIG. 9, chisel guide 150 is similar to the chisel guide 10 and generally includes a central shelf 152 having sidewalls 154 and 156. Forward wall 158 extends around the distal end of chisel guide 150 that includes all of the structure of the previously disclosed chisel guide formed with supporting surfaces, chisel guide stops and chisel stops 194, 198. In addition, the chisel guide 150 is provided with a longitudinal protrusion 160 formed midway between the side walls 154, 156 on the center shelf 152 and extending from the proximal end towards the distal end of the chisel guide 150 where it stops short of forward wall 158. Preferably, protrusion 160 has a dovetail-like cross section (not shown) to engage a similar dovetailed shape slot 204 in the associated chisel 162. Accordingly, the chisel guide 150 is configured to slidingly receive and engage the associated chisel 162, as shown in FIG. 10. Note, either FIG. 9 or FIG. 10 needs to be rotated 180 degrees to propperly engage one another. I know you've got them oriented like this just to show all the features, maybe a line in the text is needed for clarity.

The chisel 162 for use with chisel guide 150 is substantially similar to chisel 50 and includes structure to be movably attached to chisel guide 150. Chisel 162 generally includes an elongate rectangular chisel body 164 and a distal shelf 166 extending from chisel body 164. Distal shelf 166 is formed with a distal cutting edge 168. Preferably, cutting edge 168 is tapered and arcuate as shown. Additionally, in order to provide a stop to prevent the chisel from advancing too far into onto the end plate, chisel 162 is provided with sidewalls 170 having first and second portions 172 and 174. First portion 172 includes a leight H3 corresponding substantially to the height of the chisel body 164, and second portion 174 includes a height H4 less than that of height H3. The difference in heights defines a shoulder 190 configured to engage the outer surface 8 (FIG. 1) of the vertebrae and prevent over insertion of the chisel within the vertebrae. A stop 182 is formed at the distal end of second portion 174 and is configured to engage corresponding chisel stops 198, 194 formed on chisel guide 150.

As noted above, chisel 162 includes attachment structure for slidingly engaging chisel guide 150. This structure is formed on the shelf 166 and generally includes a raised elongated guide portion 202 extending longitudinally thereon and including a dovetail-shaped recess 204. Dovetail recess 204 is configured to slidingly receive protrusion 160 of chisel guide 150. Chisel guide 150 and chisel 162 are used in a manner similarly above-described with respect to chisel guide 10 in order to chisel a pocket and prepare the end plates so that their shape and dimensions would correspond the selected sape of the known spinal implant. In addition, inner surfaces of the of the sidewalls 156, 154 of the chisel guide 150 and outer sirfaces of the sides 170 can be foremd with compementary extending grooves 208 and projections 210, as shown diagrammatically, which are confgiured to prpovide additional guidance of the chisel relative to the guide 150.

It will be understood that various modifications may be made to the embodiments disclosed herein. Therefore, the above description should not be construed as limiting the scope of the invention, but merely as exemplifications of the preferred embodiments. Those skilled in the art will envision other modifications within the scope and spirit of the claims appended hereto.

What is claimed is:

1. A surgical instrumentation system for treating a disc space defined between end plates of adjacent vertebrae to be fused with spinal implants, each having a surface opposing a respective end plate and characterized by a preselected shape, comprising:
a guide frame configured to extend into the disc space; and
an endplate preparation instrument removably attached to the guide frame and controllably displaceable therealong into the disc space to provide each of the end plates with a shape corresponding to the preselected shape of the opposing surface of a respective implant.

2. The system of claim 1, wherein the guide frame includes a shelf, spaced sidewalls coupled with and flanking the shelf and juxtaposed with the end plates within the disc space, the shelf having opposite guiding surfaces slidingly supporting the end preparation instrument during displacement thereof into and out of the disc space.

3. The system of claim 2, wherein the sidewalls of the guide frame each have a distal end, the guide frame further having a forward wall bridging the distal ends of the sidewalls and coupled with the guiding surfaces of the shelf within the disc space.

4. The system of claim 3, wherein the sidewalls and the forward wall of the guide frame each have a respective pair of opposite surfaces spaced equidistantly from the shelf in opposite directions and supporting the opposite end plates.

5. The system of claim 3, wherein the guide frame further has a multiplicity of distal stops formed between the distal ends of the sidewalls and the forward wall and configured to limit penetration of the end plate preparation instrument into the disc space.

6. The system of claim 5, wherein the distal stops each extend proximally from the forward wall along a respective one of the sidewalls.

7. The system of claim 4, wherein the sidewalls of the guide frame each are formed with a proximal end extending beyond the opposite surfaces of the sidewalls in opposite directions and configured to control penetration of the guide frame into the disc space.

8. The system of claim 7, wherein the proximal ends of the sidewalls each have a distal face juxtaposed with and abutting outer surfaces of the adjacent vertebrae to provide a positive stop of the guide frame during displacement thereof into the disc space.

9. The system of claim 8, further comprising a plurality of fasteners mounted to the proximal ends of the sidewalls and extending from the distal faces thereof toward the outer surfaces of the vertebrae and attachable thereto to secure the guide frame to the vertebrae.

10. The system of claim 2, further comprising an insertion tool removably coupled to a proximal side of the shelf substantially midway between the proximal ends of the sidewalls.

11. The system of claim 10, wherein the insertion tool is screwed into the proximal side of the shelf.

12. The system of claim 3, wherein the shelf, sidewalls and the forward side of the guide frame form a monolithic body.

13. The system of claim 3, wherein at least one of the sidewalls is removably attached to the shelf and to the forward wall to remain in the disc space upon forming the pocket.

14. The system of claim 13, further comprising a guide assembly located between the at least one sidewall and the shelf of the guide frame and configured to displace the at least one sidewall and the shelf relative to one another.

15. The system of claim 14, wherein the guide assembly includes a protrusion provided on one of the at least one sidewall and the shelf and a recess provided on the other one of the at least one sidewall and the shelf.

16. The system of claim 15, wherein the recess and the protrusion are formed with mating surfaces each having a dovetail cross-section.

17. The system of claim 13, wherein the sidewalls each are removably attached to the shelf.

18. The system of claim 2, wherein the sidewalls extend either parallel or not parallel to one another and substantially perpendicular to the shelf.

19. The system of claim 2, wherein the sidewalls each have an inner surface provided with a dovetail shape.

20. The system of claim 3, wherein the forward wall of the guide frame is provided with a distally convex cross-section configured so that as the guide frame is moved into the disc space between the opposing end plates, the adjacent vertebrae are displaced from one another at a distance corresponding to a height of the sidewalls of the guide frame.

21. The system of claim 5, wherein the end plate preparation instrument includes a chisel provided with opposite faces, each having a rectangular shape, one of the opposite faces being slidably supported by a respective one of the guiding surfaces of the shelf, as the chisel moves toward the distal stops formed between the forward side and the distal ends of the sidewalls of the guide frame.

22. The system of claim 21, wherein the opposite faces extend between lateral flanks each having
a distal and proximal end,
a proximal side, bridging the proximal ends of the flanks, and
a forward edge bridging the distal ends of the flanks, the end plate preparation instrument being configured to extend beyond the forward side of the guide frame so as to uniformly remove material from each of the end plates.

23. The system of claim 22, further comprising a guiding assembly located between the end plate preparation instrument and the guide frame and configured to slidably displace the end plate preparation instrument and the guide frame relative to one another until the end plate preparation instrument comes in contact with the distal stops of the shelf of the guide frame.

24. The system of claim 22, wherein the flanks of the end plate preparation instrument each have a respective outer surface juxtaposed with an inner surface of a respective sidewall of the guide frame and extending complementary thereto, whereas the inner surfaces of the sidewalls and the outer surfaces of the flanks constitute the guiding assembly.

25. The system of claim 23, wherein one of the opposite faces of the end plate preparation instrument has a distal sunken region, the guiding assembly further including an elongated rail formed on one of the distal sunken region or on the guiding surface of the shelf and a projection formed on other one of the distal sunken region and guiding surface the shelf and having a groove.

26. The system of claim 25, wherein the groove is configured to slidably receive the elongated rail and shaped to prevent voluntary disengagement of the elongated rail during displacement of the chisel relative to the guide frame.

27. The system of claim 25, wherein the groove and the elongated rail are provided with opposing surfaces shaped to extend complementary to one another and having a U cross section or a dovetail cross section.

28. The system of claim 22, wherein the flanks of the end plate preparation instrument each have a stepwise structure including a proximal region and a distal region, which terminates short of the forward edge of the end plate preparation instrument and has an end surface abutting the distal stops of the guide frame to prevent further displacement of the end plate preparation instrument relative to the guide frame.

29. The system of claim 28, wherein the proximal regions of the flanks of end plate preparation instrument each are thicker than the distal regions and form a shoulder therewith configured to abut the outer surface of the vertebrae and to prevent further displacement of the end plate preparation instrument into the disc space.

30. The system of claim 22, wherein the forward edge of the end plate preparation instrument includes a cutting edge having an outwardly convex cross-section and is beveled.

31. The system of claim 12, wherein the shelf is hollow and configured to allow passage and removal of debris from the endplate preparation.

32. An orthopedic instrumentation system for treating opposite end plates of adjacent vertebrae defining therebetween a disc space, comprising a retractable guide frame and having
a shelf configured to extend into the disc space, and
a plurality of peripheral sides surrounding the shelf within the disc space, at least one of the peripheral sides being removably attached to the shelf to maintain a desired special position and distraction of the adjacent vertebra upon removal of the shelf from the distracted disc space; and
an endplate preparation instrument configured to slidingly engage the guide frame.

33. The system of claim 32, wherein the peripheral sides of the guide frame include spaced sidewalls extending between the end plates of the adjacent vertebra and provided with proximal stops abutting outer surfaces of the adjacent vertebra to prevent further displacement of the guide frame into the disc space.

34. The system of claim 32, further comprising a guide assembly located between the shelf of the guide frame and at least one sidewall thereof and including a channel formed on one of opposing surfaces of the at least one sidewall and the shelf and a protrusion formed on the other one of opposing surfaces, the channel and protrusion having complementary mating surfaces configured to have a U cross section or a dovetail cross section.

35. The system of claim 32, wherein the shelf has a distal end provided with spaced distal stops preventing uncontrollable displacement of the chisel within the disc space.

36. A kit for orthopedic surgery, comprising:
a guide frame configured to be inserted into a distracted disc space defined between opposing end plates of adjacent vertebrae; and
a plurality of end plate preparation instruments each configured to cooperate with the guide frame while railing therealong into and out of the disc space to treat the end plates of the adjacent vertebrae.

37. The kit of claim 36, further comprising at least one insertion tool removably coupled to the guide frame and to each of the plurality of end preparation instrument to provide displacement thereof into and out of the disc space.

38. The kit of claim 37, wherein the guide frame includes a shelf extending parallel to the end plates after the guide frame is inserted into the disc space and a plurality of sidewalls extending along a periphery of the shelf and selectively detachable therefrom, wherein the at least one insertion tool is selectively coupled to the shelf and to the sidewalls to provide displacement of the sidewalls and the shelf relative to one another from the disc space.

39. The kit of claim 38, wherein the sidewalls of the guide frame each have a proximal end extending from the shelf in opposite direction to abut an outer surface of a respective vertebrae to prevent a further displacement of the guide frame into the disc space, a distal end of each of the sidewalls being provided with a distal stop extending laterally towards and terminating at a distance from the other sidewall of the guide frame to prevent uncontrollable displacement of the end plate preparation instrument

40. The kit of claim 39, wherein the proximal stops of the guide frame are provided with a plurality of fasteners configured to engage the outer surface of the adjacent vertebrae.

41. The kit of claim 39, wherein inner surfaces of the sidewalls, facing one another, each slidingly engage a respective one of opposite flanks of each of the end plate preparation instrument, wherein the engageable inner surfaces and the flanks have complementary extending mating projection and recess providing displacement of the end plate preparation instrument into and out of the disc space.

42. The kit of claim 39, wherein the shelf has opposite faces each opposing to a respective end plate and providing a support surface for the end plate preparation instruments during displacement thereof into and out of the disc space, at least one of the opposite faces of the shelf and a side of each of the end plate preparation instruments, juxtaposed with the at least one face of the shelf during displacement of the end preparation instruments, having complementary shaped and mating formations training the end plate preparation instruments into and out of the disc space.

43. The kit of claim 39, wherein the end plate preparation instruments each have a pair of leading stops abutting the distal stops of the guide frame to prevent further displacement of the end plate preparations instruments into the disc space.

44. The kit of claim 36, further comprising a plurality of variously shaped and dimensioned spinal implants.

45. The kit of claim 44, further comprising an implant insertion holder selectively attachable to the plurality of spinal implants.

46. The kit of claim 37, further comprising a slap hammer configured to engage the at least one insertion, the slap hammer having an elongated shaft removably attachable to the at least one insertion instrument and a weight displaceably mounted on the elongated shaft and movable towards a proximal end of the slap hammer to facilitate removal of the guide frame from the disc space.

47. A method of creating a space of a selected shape across a disc space between opposing end plates of adjacent vertebrae to be fused with a spinal implant, comprising the steps of:
introducing a guide frame provided with an H-shape into the disk space so that spaced sidewalls of the guide frame support the opposing end plates;
guidingly displacing an end plate preparation instrument along one of opposite faces of a shelf, extending between the sidewalls of the guide frame, and into one of the end plates of the adjacent vertebrae, whereas the end plate preparation instrument removes a desired amount of material from the one end plate while being displaced into the disc space; and
guidingly displacing the end plate preparation instrument along the other one of opposite faces of the shelf, thereby removing the desired amount of material from the other end plate, whereas the opposing end plates of the adjacent vertebrae define therebetween the space of the selected shape corresponding to the shape of the spinal implant.

47. The method of claim 46, further comprising the step of controlling penetration of the of guide frame into the disc space.

48. The method of claim 46, further comprising the step of controllably stopping displacement of the end plate preparation instrument relative to the guide frame after the desired amount of material has been from each of the opposing end plates.

49. The method of claims 46, further comprising the steps of piecemeal removal of the sidewalls and the shelf from the disc shape after the selected shape has been applied thereto, and inserting the spinal implant.
